(19) United States
(12) Patent Application Publication
(10) Pub. No.: US 2015/0108447 A1
(43) Pub. Date: Apr. 23, 2015

(54) DIOXAANTHANTHRENE COMPOUND AND ELECTRONIC DEVICE

(71) Applicant: Sony Corporation, Tokyo (JP)

(72) Inventors: Norihito Kobayashi, Kanagawa (JP); Eri Igarashi, Kanagawa (JP)

(73) Assignee: Sony Corporation, Tokyo (JP)

(21) Appl. No.: 14/402,814

(22) PCT Filed: May 2, 2013
(86) PCT No.: PCT/JP2013/062773

§ 371 Date: Nov. 21, 2014

(30) Foreign Application Priority Data
May 29, 2012 (JP) 2012-121835

(51) Int. Cl. H01L 51/00; C07D 493/06

(52) U.S. Cl. CPC H01L 51/0073; C07D 493/06; H01L 51/0058; H01L 51/0541
USPC 257/40; 549/381

(57) ABSTRACT

Provided is a dioxaanthanthrene compound represented by the following structural formula (1).

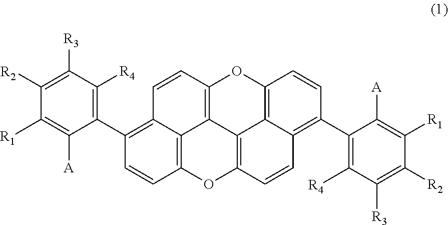

### TECHNICAL FIELD

The present disclosure relates to a dioxaanthanthrene compound, and an electronic device that includes an active layer formed from such a dioxaanthanthrene compound.

### BACKGROUND ART

Recently, semiconductor devices that include a semiconductor layer formed from an organic semiconductor material are drawing a lot of attention. Such a semiconductor device allows a semiconductor layer to be coated and formed at a lower temperature than a structure that includes a semiconductor layer formed from an inorganic material. Consequently, not only is there the advantage of a larger surface area, but plastics and other such materials that have low heat resistance but are flexible can be formed on a substrate, leading to the expectation of increased functionality as well as reduced costs.

Currently, for example, polyacene compounds, such as anthracene, naphthacene, and pentacene, having the following structural formula are being widely researched as an organic semiconductor material forming a semiconductor layer.

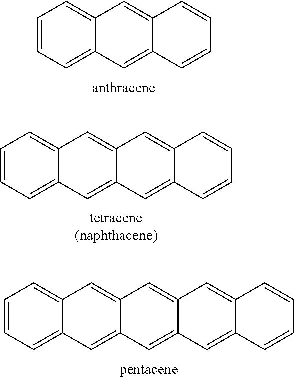

Further, in JP 2010-006794A, the present inventors proposed an organic semiconductor material (specifically, a dioxaanthanthrene compound) that exhibits a high carrier mobility, as well as a high level of freedom in molecular design and high process adaptability, and an electronic device that includes a semiconductor layer formed from such an organic semiconductor material.

### CITATION LIST

### Patent Literature

Patent Literature 1: JP 2010-006794A

### SUMMARY OF INVENTION

### Technical Problem

Although the dioxaanthanthrene compound disclosed in this patent publication exhibits high carrier mobility, as well as a high level of freedom in molecular design and high process adaptability, there is a need for higher solubility.

Therefore, it is an object of the present disclosure to provide a dioxaanthanthrene compound that exhibits higher solubility and an electronic device that includes an active layer formed from such a dioxaanthanthrene compound.

According to the first aspect of the present disclosure in order to achieve the above-mentioned object, there is provided a dioxaanthanthrene compound represented by the following structural formula (1),

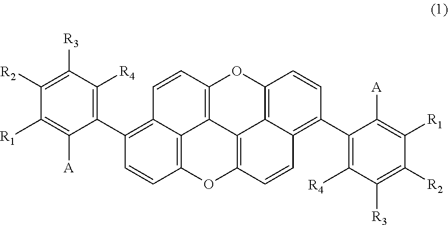

wherein a substituent A is one kind of substituent selected from the group consisting of an alkyl group, a cycloalkyl group, an alkenyl group, an alkynyl group, an aryl group, an arylalkyl group, an aromatic heterocycle, a heterocyclic group, an alkoxy group, a cycloalkoxy group, an aryloxy group, an alkylthio group, a cycloalkylthio group, an arylthio group, an alkoxycarbonyl group, an aryloxycarbonyl group, a sulfamoyl group, an acyl group, an acyloxy group, an amide group, a carbamoyl group, a ureido group, a sulfinyl group, an alkylsulfonyl group, an arylsulfonyl group, an amino group, a halogen atom, a fluorinated hydrocarbon group, a cyano group, a nitro group, a hydroxy group, a mercapto group, and a silyl group including a trimethyl silyl group, and

wherein substituents R₁, R₂, R₃, and R₄ are each independently a hydrogen atom or one kind of substituent selected from the group consisting of an alkyl group, a cycloalkyl group, an alkenyl group, an alkynyl group, an aryl group, an arylalkyl group, an aromatic heterocycle, a heterocyclic group, an alkoxy group, a cycloalkoxy group, an aryloxy group, an alkylthio group, a cycloalkylthio group, an arylthio group, an alkoxycarbonyl group, an aryloxycarbonyl group, a sulfamoyl group, an acyl group, an acyloxy group, an amide group, a carbamoyl group, a ureido group, a sulfinyl group, an alkylsulfonyl group, an arylsulfonyl group, an amino group, a halogen atom, a fluorinated hydrocarbon group, a cyano group, a nitro group, a hydroxy group, a mercapto group, and a silyl group including a trimethyl silyl group.

According to the second aspect of the present invention in order to achieve the above-mentioned object, there is provided a dioxaanthanthrene compound formed of 3,9-diphenyl peri-xanthenoxanthene (which may be abbreviated as “Ph-PXX”), in which at least an ortho-position of two phenyl groups is substituted with a substituent A,

wherein the substituent A is one kind of substituent selected from the group consisting of an alkyl group, a cycloalkyl group, an alkenyl group, an alkynyl group, an aryl group, an arylalkyl group, an aromatic heterocycle, a heterocyclic group, an alkoxy group, a cycloalkoxy group, an aryloxy group, an alkylthio group, a cycloalkylthio group, an arylthio group, an alkoxycarbonyl group, an aryloxycarbonyl group, a sulfamoyl group, an acyl group, an acyloxy group, an amide group, a carbamoyl group, a ureido group, a sulfinyl group, an alkylsulfonyl group, an arylsulfonyl group, an amino group, a halogen atom, a fluorinated hydrocarbon group, a cyano group, a nitro group, a hydroxy group, a mercapto group, and a silyl group including a trimethyl silyl group.

An electronic device in order to achieve the above-mentioned object includes at least:

a first electrode;

a second electrode disposed separated from the first electrode; and

an active layer provided from the first electrode to the second electrode,

wherein the active layer is formed of the dioxaanthanthrene compound according to any one of the first and second aspects of the present disclosure.

### Solution to Problem

### Advantageous Effects of Invention

Since the dioxaanthanthrene compound according to the first and second embodiments of the present disclosure is formed from a dioxaanthanthrene compound represented by structural formula (1), or alternatively, Ph-PXX in which at least an ortho-position of two phenyl groups is substituted with a substituent A, a high solubility can be obtained.

### BRIEF DESCRIPTION OF DRAWINGS

FIGS. 1A and 1B are schematic partial end diagrams of a base and the like for illustrating an outline of the method for manufacturing the electronic device of Working Example 3.

FIGS. 2A and 2B are schematic partial end diagrams of a base and the like for illustrating an outline of the method for manufacturing the electronic device of Working Example 4.

FIGS. 3A and 3B are schematic partial end diagrams of a base and the like for illustrating an outline of the method for manufacturing the electronic device of Working Example 5.

FIGS. 4A, 4B, and 4C are schematic partial end diagrams of a base and the like for illustrating an outline of the method for manufacturing the electronic device of Working Example 6.

FIGS. 5A and 5B are schematic partial end diagrams of a base and the like for illustrating an outline of the method for manufacturing the electronic device of Working Example 7.

### DESCRIPTION OF EMBODIMENTS

Hereinafter, preferred embodiments of the present invention will be described in detail with reference to the appended drawings. Note that, in this specification and the drawings, elements that have substantially the same function and structure are denoted with the same reference signs, and repeated explanation is omitted. Further, although the present disclosure will be described below based on working examples with reference to the drawings, the present disclosure is not limited to the working examples, and the various numerical values and materials in the working examples are illustrative. The description will now be made in the following order.

1. Dioxaanthanthrene compound according to the first and second embodiments of the present disclosure, electronic device, and overall description
2. Working Example 1 (dioxaanthanthrene compound according to the first and second embodiments of the present disclosure)
3. Working Example 2 (modification of Working Example 1)
4. Working Example 3 (electronic device according to the present disclosure)
5. Working Example 4 (modification of Working Example 3)
6. Working Example 5 (another modification of Working Example 3)
7. Working Example 6 (yet another modification of Working Example 3)
8. Working Example 7 (electronic device according to the present disclosure, a two-terminal type electronic device), and other matters

### [Dioxaanthanthrene Compound According to the First and Second Embodiments of the Present Disclosure, Electronic Device, and Overall Description]

The electronic device according to the present disclosure can be configured as an embodiment that includes a first electrode, a second electrode disposed separated from the first electrode, a control electrode, and an insulating layer,

wherein the control electrode is provided facing a portion of an active layer that is positioned between the first electrode and the second electrode via the insulating layer, namely, as a so-called three-terminal type electronic device. However, the electronic device according to the present disclosure is not limited to this, and can also be configured as a so-called two-terminal type electronic device.

The dioxaanthanthrene compound according to the first embodiment of the present disclosure, or, the dioxaanthanthrene compound constituting the active layer in the electronic device that includes the above-described preferred modes (hereinafter, these dioxaanthanthrene compounds are sometimes collectively referred to as “the dioxaanthanthrene compounds according to the first embodiment of the present disclosure”), may be configured so that

a substituent A is one kind of substituent selected from the group consisting of an alkyl group, an alkenyl group, an aryl group, an arylalkyl group, an aromatic heterocycle, and a halogen atom, and

substituents R₁, R₂, R₃, and R₄ are each independently a hydrogen atom or one kind of substituent selected from the group consisting of an alkyl group, an alkenyl group, an aryl group, an arylalkyl group, an aromatic heterocycle, and a halogen atom. Alternatively, the dioxaanthanthrene compound according to the first embodiment of the present disclosure may be configured so that

the substituent A is an alkyl group,

the substituents R₁, R₂, R₃, and R₄ are each independently a hydrogen atom or the same alkyl group as the substituent A, and

a solubility (solubility ratio) of the dioxaanthanthrene compound represented by structural formula (1) is three times or more that of the solubility of the dioxaanthanthrene compound (Ph-PXX in which a para-position of two phenyl groups is substituted with the substituent A) represented by structural formula (2). It is noted that the measurement of the solubility of the dioxaanthanthrene compound is carried out at 25° C. using toluene or xylene as a solvent.

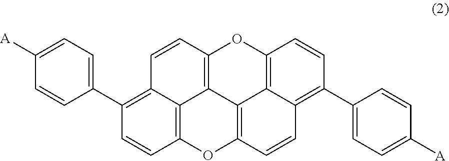

The dioxaanthanthrene compound according to the second embodiment of the present disclosure, or, the dioxaanthanthrene compound constituting the active layer in the electronic device that includes the above-described preferred modes (hereinafter, these dioxaanthanthrene compounds are sometimes collectively referred to as “the dioxaanthanthrene compounds according to the second embodiment of the present disclosure”), may be configured so that an ortho-position of two phenyl groups is substituted with the substituent A, or alternatively, may be configured so that an ortho-position and a para-position of two phenyl groups, or an ortho-position and a meta-position of two phenyl groups is substituted with the substituent A.

A preferred relationship of substituents R₁ to R₄ of the dioxaanthanthrene compound according to the first embodiment of the present disclosure is listed below in Table 1. It is noted that in cases 6 to 9, the two substituents A may be the same or different.

TABLE 1

| Case | R₁ | R₂ | R₃ | R₄ |
|---|---|---|---|---|
| 1 | Hydrogen Atom | Hydrogen Atom | Hydrogen Atom | Hydrogen Atom |
| 2 | Substituent A | Hydrogen Atom | Hydrogen Atom | Hydrogen Atom |
| 3 | Hydrogen Atom | Substituent A | Hydrogen Atom | Hydrogen Atom |
| 4 | Hydrogen Atom | Hydrogen Atom | Substituent A | Hydrogen Atom |
| 5 | Hydrogen Atom | Hydrogen Atom | Hydrogen Atom | Substituent A |
| 6 | Substituent A | Substituent A | Hydrogen Atom | Hydrogen Atom |
| 7 | Substituent A | Hydrogen Atom | Substituent A | Hydrogen Atom |
| 8 | Substituent A | Hydrogen Atom | Hydrogen Atom | Substituent A |
| 9 | Hydrogen Atom | Substituent A | Hydrogen Atom | Substituent A |

In the dioxaanthanthrene compound according to the first embodiment of the present disclosure including the above-mentioned preferable configuration, or, the dioxaanthanthrene compound according to the second embodiment of the present disclosure (hereinafter, these are sometimes collectively referred to simply as “the dioxaanthanthrene compounds etc. according to the embodiments of the present disclosure”), examples of the alkyl group include a methyl group, an ethyl group, a propyl group, an isopropyl group, a tertiary butyl group, a pentyl group, a hexyl group, an octyl group, a dodecyl group and the like. It is noted that the alkyl group may be linear or branched. Further, examples of the cycloalkyl group may include a cyclopentyl group, a cyclohexyl group and the like; examples of the alkenyl may include a vinyl group and the like; examples of the alkynyl group may include an ethynyl group; examples of the aryl group may include a phenyl group, a naphthyl group, a biphenyl group and the like; examples of the arylalkyl group may include a methyl aryl group, an ethyl aryl group, an isopropyl aryl group, normal butyl aryl group, a p-tolyl group, a p-ethylphenyl group, a p-isopropylphenyl group, a p-iso-butylphenyl group, a 4-propylphenyl group, a 4-butylphenyl group, a 4-nonylphenyl group and the like; examples of the aromatic heterocycle may include a pyridyl group, a thienyl group, a furyl group, a pyridazinyl group, a pyrimidinyl group, a pyrazinyl group, a triazinyl group, an imidazolyl group, a pyrazolyl group, a thiazolyl group, a quinazolinyl group, a phthalazinyl group and the like; examples of the heterocyclic group may include a pyrrolidyl group, an imidazolidyl group, a morpholinyl group, an oxazolidyl group and the like; examples of the alkoxy group may include a methoxy group, an ethoxy group, a propyloxy group, a pentyloxy group, a hexyloxy group and the like; examples of the cycloalkoxy group may include a cyclopentyl group, a cyclohexyl group and the like; examples of the aryloxy group may include a phenoxy group, a naphthyloxy group and the like; examples of the alkylthio group may include a methylthio group, an ethylthio group, a propylthio group, a pentylthio group, a hexylthio group and the like; examples of the cycloalkylthio group may include a cyclopentylthio group, a cyclohexylthio group and the like; examples of the arylthio group may include a phenylthio group, a naphthylthio group and the like; examples of the alkoxycarbonyl group may include a methyloxycarbonyl group, an ethyloxycarbonyl group, a butyloxycarbonyl group, an octyloxycarbonyl group and the like; examples of the aryloxycarbonyl group may include a phenyloxycarbonyl group, a naphthyloxycarbonyl group and the like; examples of the sulfamoyl group may include an aminosulfonyl group, a methylaminosulfonyl group, a dimethylaminosulfonyl group, a cyclohexylaminosulfonyl group, a phenylaminosulfonyl group, a naphthylaminosulfonyl group, a 2-pyridylaminosulfonyl group and the like; examples of the acyl group may include an acetyl group, an ethylcarbonyl group, a propylcarbonyl group, a cyclohexylcarbonyl group, an octylcarbonyl group, a 2-ethylhexylcarbonyl group, a dodecylcarbonyl group, a phenylcarbonyl group, a naphthylcarbonyl group, a pyridylcarbonyl group and the like; examples of the acyloxy group may include an acetyloxy group, an ethylcarbonyloxy group, an octylcarbonyloxy group, a phenylcarbonyloxy group and the like; examples of the amide may include a methylcarbonylamino group, an ethylcarbonylamino group, a dimethylaminocarbonylamino group, a pentylcarbonylamino group, a cyclohexylcarbonylamino group, 2-ethylhexylcarbonylamino group, a phenylcarbonylamino group, a naphthylcarbonylamino group and the like; examples of the carbamoyl group may include an aminocarbamoyl group, a methylaminocarbamoyl group, a dimethylaminocarbamoyl group, a cyclohexylaminocarbamoyl group, a 2-ethylhexylaminocarbamoyl group, a phenylaminocarbamoyl group, a naphthylaminocarbamoyl group, a 2-pyridylaminocarbonyl group and the like; examples of the ureido group may include a methylureido group, an ethylureido group, a cyclohexylureido group, a dodecylureido group, a phenylureido group, a naphthylureido group, a 2-pyridylaminoureido group and the like; examples of the sulfinyl may include a methylsulfinyl group, an ethylsulfinyl group, a butylsulfinyl group, a cyclohexyl sulfinyl group, a 2-ethylhexylsulfinyl group, a phenylsulfinyl group, a naphthylsulfinyl group, a 2-pyridylsulfinyl group and the like; examples of the alkylsulfonyl group may include a methylsulfonyl group, an ethylsulfonyl group, a butylsulfonyl group, a cyclohexylsulfonyl group, a 2-ethylhexylsulfonyl group, a dodecylsulfonyl group and the like; examples of the arylsulfonyl group may include a phenylsulfonyl group, a naphthylsulfonyl group, a 2-pyridylsulfonyl group and the like; examples of the amino group may include an amino group, an ethylamino group, a dimethylamino group, a butylamino group, a 2-ethylhexylamino group, an anilino group, a naphthylamino group, a 2-pyridylamino group and the like; examples of the halogen atom may include a fluorine atom, a chlorine atom, a bromine atom, an iodine atom and the like; and examples of the fluorinated hydrocarbon group may include a fluoromethyl group, a trifluoromethyl group, a pentafluoroethyl group, a pentafluorophenyl group and the like. Further examples may also include a cyano group, a nitro group, a hydroxy group, a mercapto group, a silyl group and the like. Examples of the silyl group a trimethylsilyl group, a triisopropylsilyl group, a triphenylsilyl group, a phenyl group diethylsilyl and the like. Here, the above-described substituents may be further substituted with an above-described substituent. Further, a plurality of these substituents may be joined to each other to form a ring. In addition, an additive (e.g., a so-called doping material, such as an n-type impurity or a p-type impurity) can also be added. Note that the additive may be separately added to the dioxaanthanthrene compound and the like of the present disclosure, or may be added in a form in which it is included on a substituent.

The electronic device according to the present disclosure may have a so-called three-terminal structure, or a two-terminal structure. Further, for example, a field-effect transistor (FET), specifically, a thin-film transistor (TFT), is configured by an electronic device having such a three-terminal structure, or alternatively, a light emitting element is configured by an electronic device having such a three-terminal structure. Namely, a light emitting element (an organic light emitting element, an organic light emitting transistor) in which an active layer emits light based on the application of a voltage to the control electrode and the first electrode and second electrode can be configured. In these electronic devices, the current flowing in the active layer from the first electrode to the second electrode is controlled based on the voltage applied to the control electrode. Here, in the light emitting element, the dioxaanthanthrene compound constituting the active layer has a light-emitting function based on charge storage due to modulation based on the voltage applied to the control electrode and recombination of the injected electrons and holes. Emission intensity, which is proportional to the absolute value of the current flowing from the first electrode to the second electrode, can be modulated the voltage applied to the control electrode and the voltage applied between the first electrode and the second electrode. Whether the electronic device exhibits a function as a field-effect transistor or as a light emitting element depends on the state (bias) of voltage application to the first and second electrodes. First, when the control electrode is modulated under a condition in which a bias is applied in a range where electrons are not injected from the second electrode, a current flows from the first electrode to the second electrode. This is a transistor operation. On the other hand, when the bias to the first electrode and the second electrode is increased under a condition in which holes have been sufficiently stored, electron injection starts, and light is emitted based on the recombination with holes. Further, an example of an electronic device having a two-terminal structure includes a photoelectric conversion element in which current flows between the first electrode and the second electrode by irradiation of light on the active layer. If a photoelectric conversion element is configured by the electronic device, specifically, a solar cell or various sensors, such as an image sensor or a light sensor, can be configured by the photoelectric conversion element. Alternatively, the electronic device can configure an organic electroluminescence element (organic EL element) or an organic EL display device, and can function as a chemical substance sensor. Namely, the electronic device can be used in a mode as a display element, a display device, a solar cell, or a sensor. It is noted that the photoelectric conversion element can also be configured from an electronic device having a three-terminal structure. In this case, a voltage may or may not be applied to the control electrode. If a voltage is applied, the current that is flowing can be modulated based on the application of the voltage to the control electrode. Further, the light emitting part of the organic EL element can also be configured by the dioxaanthanthrene compound according to the first to third embodiments of the present disclosure.

The first electrode and second electrode, and the active layer are formed on the base, or alternatively, above the base.

In the case of configuring a semiconductor device from the electronic device of the present disclosure, specific examples of the semiconductor device include a bottom-gate/bottom-contact type field-effect transistor (FET), a bottom-gate/top-contact type FET, a top-gate/bottom-contact type FET, and a top-gate/top-contact type FET.

If the semiconductor device is configured by a bottom-gate/bottom-contact type field-effect transistor (FET), this bottom-gate/bottom-contact type FET includes

(A) a gate electrode (control electrode) formed on a base,

(B) a gate insulating layer (insulating layer) formed on the gate electrode and the base,

(C) source/drain electrodes (first electrode and second electrode) formed on the gate insulating layer, and

(D) a channel formation region configured by an active layer, which is formed on the gate insulating layer between the source/drain electrodes.

Alternatively, if the semiconductor device is configured by a bottom-gate/top-contact type FET, this bottom-gate/top-contact type FET includes

(A) a gate electrode (control electrode) formed on a base,

(B) a gate insulating layer (insulating layer) formed on the gate electrode and the base,

(C) a channel formation region and a channel formation region extension portion which are formed on the gate insulating layer and are configured by an active layer, and

(D) source/drain electrodes (first electrode and second electrode) formed on the channel formation region extension portion.

Alternatively, if the semiconductor device is configured by a top-gate/bottom-contact type FET, this top-gate/bottom-contact type FET includes

(A) source/drain electrodes (first electrode and second electrode) formed on a base,

(B) a channel formation region which is formed on the base between the source/drain electrodes and is configured by an active layer,

(C) a gate insulating layer (insulating layer) formed on the source/drain electrodes and the channel formation region, and

(D) a gate electrode (control electrode) formed on the gate insulating layer.

Alternatively, if the semiconductor device is configured by a top-gate/top-contact type FET, this top-gate/top-contact type FET includes

(A) a channel formation region and a channel formation region extension portion which are formed on a base and are configured by an active layer,

(B) source/drain electrodes (first electrode and second electrode) formed on the channel formation region extension portion,

(C) a gate insulating layer (insulating layer) formed on the source/drain electrodes and the channel formation region, and

(D) a gate electrode (control electrode) formed on the gate insulating layer.

Here, the base can be configured by a silicon oxide-based material (e.g., SiOₓ, spin-on glass (SOG), silicon oxynitride (SiON)); silicon nitride (SiN_{Y}); a metal oxide high-dielectric insulating film, such as aluminum oxide (Al₂O₃) and HfO₂; metal oxides; and metal salts. If the base is configured by these materials, the base may be formed on a support (or above a support) appropriately selected from among the materials listed below. Namely, examples of the support, or alternatively, a base other than the above-described base, include organic polymers (in the form of a polymer material of a flexible plastic film, a plastic sheet, or a plastic substrate configured by a polymer material), such as polymethylmethacrylate (PMMA), polyvinyl alcohol (PVA), polyvinyl phenol (PVP), polyether sulfone (PES), polyimide, polycarbonate (PC), polyethylene terephthalate (PET), polyethylene naphthalate (PEN), and the like. Alternatively, examples may include natural mineral-based insulating materials, such as mica, metal-based semiconductor materials, molecular semiconductor materials and the like. If a base configured by such a flexible polymer material is used, for example, the electronic device can be mounted on or integrated with an image display device (display device) or electronic equipment having a curved surface shape. Alternatively, further examples of the base include various glass substrates, various glass substrates in which an insulating film is formed on the surface, a quartz substrate, a quartz substrate in which an insulating film is formed on the surface, a silicon substrate in which an insulating film is formed on the surface, and a conductive substrate (metals such as gold, aluminum, and stainless steel, a substrate configured by alloys, a substrate including highly-oriented graphite) in which an insulating film is formed on the surface. As the support having an electrical insulating property, a suitable material may be selected from among the above-described materials. Further examples of the support include a conductive substrate (a substrate including a metal such as gold and aluminum, a substrate including highly-oriented graphite, a stainless steel substrate etc.). In addition, depending on the mode and structure of the electronic device, the electronic device may be disposed on a support member, and this support member may be configured by the above-described materials.

Examples of the material constituting the control electrode, first electrode, second electrode, gate electrode, source/drain electrodes, and wiring (hereafter, these are collectively referred to as “control electrode etc.”) include metals, such as platinum (Pt), gold (Au), palladium (Pd), chromium (Cr), nickel (Ni), aluminum (Al), silver (Ag), tantalum (Ta), tungsten (W), copper (Cu), titanium (Ti), indium (In), tin (Sn), iron (Fe), cobalt (Co), zinc (Zn), magnesium (Mg), manganese (Mn), ruthenium (Rh), a rubidium (Rb), and molybdenum (Mo), or, conductive substances, such as an alloy including these metals elements, conductive particles including these metals, conductive particles including an alloy of these metals, polysilicon containing impurities, a carbon material and the like. A laminated structure layers including these elements can also be used. Further examples of the material constituting the control electrode etc. include an organic material (conductive polymer), such as poly(3,4-ethylenedioxythiophene)/polystyrene sulfonate [PEDOT/PSS], TTF-TCNQ, and poly aniline. The materials which constitute the control electrode etc. may be the same material or a different material.

Although the method for forming the control electrode etc. depends on the materials constituting these parts, examples may include a physical vapor deposition method (PVD method); pulsed laser deposition (PLD), an arc discharge method; various chemical vapor deposition methods including an MOCVD method; a spin coating method; various printing methods, such as a screen printing method, an ink jet printing method, an offset printing method, a reverse offset printing method, a gravure printing method, a gravure offset printing method, relief printing, flexo printing, and a micro contact method; various coating methods, such as an air doctor coater method, a blade coater method, a rod coater method, a knife coater method, a squeeze coater method, a reverse roll coater method, a transfer roll coater method, a gravure coater method, a kiss coater method, a cast coater method, a spray coater method, a slit coater method, a slit orifice coater method, a calender coater method, a casting method, a capillary coater method, a bar coater method, and a dipping method; a stamp method; a casting method; a method using a dispenser; a spray method; a lift-off method; a shadow mask method; as well as a combination of any plating method, such as an electrolytic plating method, an electroless plating method, or a combination thereof, with optionally a patterning technique. Examples of the PVD method include (a) an electron beam heating method, a resistance heating evaporation method, various vacuum deposition methods, such as flash evaporation, a method of heating a crucible and the like (b) a plasma evaporation method, (c) various sputtering methods, such as a diode sputtering method, a direct-current sputtering method, a direct-current magnetron sputtering method, a high-frequency sputtering method, a magnetron sputtering method, an ion beam sputtering method, a bias sputtering method and the like, and (d) various ion ion plating methods, such as a DC (direct current) method, a RF method, a multi-cathode method, an activation reaction method, a field evaporation method, a high-frequency ion plating method, a reactive ion plating method and the like. When the control electrode etc. are formed based on an etching method, a dry-etching method or a wet-etching method may be employed. Examples of dry-etching methods include ion milling and reactive ion etching (RIE). Further, the control electrode etc. may also be formed based on a laser ablation method, a mask evaporation method, a laser transfer method and the like.

Examples of the material constituting the insulating layer (the gate insulating layer) not only include an inorganic insulating material, such as a silicon oxide-based material; silicon nitride (SiN_{Y}); and a metal oxide high-dielectric insulating film, such as aluminum oxide (Al₂O₃) and HfO₂, but also an organic insulating material (organic polymer), such as a straight-chain hydrocarbon having on one end a functional group that can be bonded to the control electrode etc. (the gate electrode), such as polymethylmethacrylate (PMMA); polyvinyl phenol (PVP); polyvinyl alcohol (PVA); polyimide; polycarbonate (PC); polyethylene terephthalate (PET); polystyrene; a silanol derivative (silane coupling agent) such as N-2(aminoethyl)-3-aminopropyltrimethoxysilane (AEAPTMS), 3-mercaptopropyltrimethoxysilane (MPTMS), or octadecyltrichlorosilane (OTS); octadecanethiol; and dodecyl isocyanate). A combination of these may also be used. Here, examples of the silicon oxide-based material include oxidized silicon (SiO_{X}), BPSG, PSG, BSG, AsSG, PbSG, silicon oxynitride (SiON), SOG (spin-on glass), or a low-permittivity material (e.g., polyarylether, cycloperfluorocarbon polymer and benzocyclobutene, a cyclic fluororesin, an amorphous resin (e.g., CYTOP manufactured by Asahi Glass Co., Ltd.), polytetrafluoroethylene, fluorinated aryl ether, fluorinated polyimide, amorphous carbon, and organic SOG).

The insulating layer (gate insulating layer) can be formed by the above-described various PVD methods; various CVD methods, a spin coating method; the above-described various printing methods; the above-described various coating methods; a dipping method; a casting method; a sol-gel method; an electrodeposition method; a shadow mask method; as well as any spray method. Alternatively, the insulating layer can also be formed by oxidizing or nitriding the surface of the control electrode (gate electrode), or obtained by forming an oxide film or a nitride film on the surface of the control electrode. Although the method of oxidizing the surface of the control electrode depends on the material constituting the control electrode, examples may include an oxidizing method using O₂ plasma or an anodization method. Further, although the method of nitriding the surface of the control electrode depends on the materials constituting the control electrode, examples may include a nitriding method using N₂ plasma. Alternatively, for an Au electrode, the insulating layer (gate insulating layer) can also be formed on the surface of the control electrode (the gate electrode) by, for example, covering the control electrode surface in a self-organizing manner by a method such as a dipping method with insulating molecules having a functional group capable of forming a chemical bond with the control electrode, like a linear hydrocarbon in which one end is modified by a mercapto group. Alternatively, the insulating layer (the gate insulating layer) may be formed by modifying the surface of the control electrode (the gate electrode) with a silanol derivative (silane coupling agent).

Examples of the dioxaanthanthrene compounds according to the present disclosure, the active layer, or the channel formation region and the channel formation region extension portion, include, but are not limited to, a spin coating method; the above-described various printing methods; the above-described various coating methods; a stamping method; a casting method; a method using a dispenser; and a wet deposition method such as a spraying method. The active layer can also optionally be patterned by a known method such as, for example, a wet etching method, a dry etching method, or a laser ablation method. A known solvent selected as appropriate may be used as the solvent for dissolving the dioxaanthanthrene compounds according to the present disclosure. Specific examples include at least one kind selected from xylene, p-xylene, o-xylene, mesitylene, toluene, tetralin, anisole, benzene, 1,2-dichlorobenzene, o-dichlorobenzene, cyclohexane, and ethyl cyclohexane. The drying conditions (the time, temperature etc.) of the solution in which the dioxaanthanthrene compounds according to the present disclosure is dissolved can be appropriately determined based on the used solvent and the like.

Examples of devices in which the electronic device according to the present disclosure is mounted may include, for example, an image display device. Here, examples of an image display device may include a so-called desktop type personal computer, a notebook type personal computer, a mobile type personal computer, a PDA (personal digital assistant), a mobile phone, a game machine, electronic paper such as an electronic book and an electronic newspaper, a message board such as a signboard, a poster, and a blackboard, a copy machine, rewritable paper to substitute for printer paper, a calculator, a display unit in household appliances, a card display unit such as a point card, and various image display devices in electronic advertizing and electronic POP (e.g., an organic electroluminescence display device, a liquid crystal display device, a plasma display device, an electrophoretic display device, a cold cathode field emission display device etc.). Further examples include various lighting apparatuses.

If the electronic device is applied or used in various image display devices or various electronic machines, the used electronic device may be used as a monolithic integrated circuit in which multiple electronic devices have been integrated on a support member, or each electronic device may be individually separated and used as a discrete component. Further, the electronic device may be sealed with a resin.

### Working Example 1

Working Example 1 relates to the dioxaanthanthrene compound according to the first and second embodiments of the present disclosure.

The dioxaanthanthrene compound of Working Example 1 is, specifically, formed from 3,9-diphenyl peri-xanthenoxanthene (Ph-PXX), in which the substituent A in the above structural formula (1) is an alkyl group, and substituents R₁ to R₄ are hydrogen atoms. Alternatively, the dioxaanthanthrene compound of Working Example 1 is, specifically, formed from the 3,9-diphenyl peri-xanthenoxanthene represented by the following structural formula (3), in which, at least an ortho-position of two phenyl groups (in Working Example 1, specifically, an ortho-position) is substituted with a substituent A formed from an alkyl group.

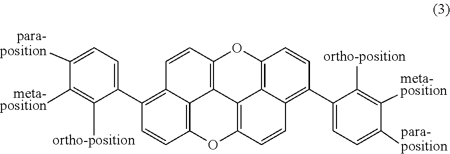

It is noted that such a dioxaanthanthrene compound can be obtained by reacting peri-xanthenoxanthene and bromine to obtain 3,9-dibromo peri-xanthenoxanthene, and then reacting the bromine atoms with phenylboronic acid having an alkyl group at an ortho-position.

The dioxaanthanthrene compound of Working Example 1A is, specifically, formed from the compound (o-iC1Ph-PXX) represented by formula (11), and the dioxaanthanthrene compound of Working Example 1A is, specifically, formed from the compound (o-iC4Ph-PXX) represented by formula (12). The solubility at 25° C. in 100 g of toluene is as shown in the following Table 2. It is noted that the dioxaanthanthrene compound of Comparative Example 1A is p-iC1Ph-PXX, and the dioxaanthanthrene compound of Comparative Example 1B is p-iC4Ph-PXX.

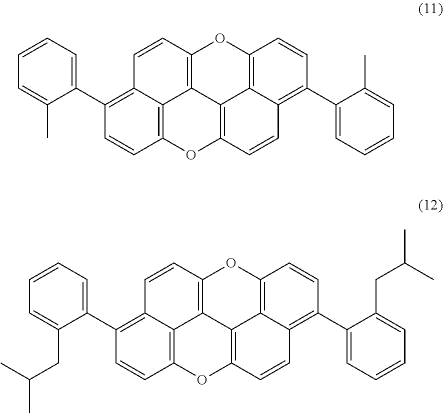 [SMILES: Cc1ccccc1-c1ccc2oc3ccc4c(-c5ccccc5C)ccc5oc6ccc1c2c6-c3c54 ; CC(C)Cc1ccccc1-c1ccc2oc3ccc4c(-c5ccccc5CC(C)C)ccc5oc6ccc1c2c6-c3c54]

TABLE 2

| Solubility (grams) | | Solubility Ratio |
|---|---|---|
| Working Example 1A | 0.60 | 5.0 (Working Example 1A/ |
| Comparative Example 1 | 0.12 | Comparative Example 1A) |
| Working Example 1B | 20 | 10 (Working Example 1B/ |
| Comparative Example 1B | 2 | Comparative Example 1B) |
| Working Example 2 | 5.4 | 45 (Working Example 2/ |
| | | Comparative Example 1A) |

The dioxaanthanthrene compound of Working Example 1 or the below-described Working Example 2 has, as described above, a high solubility.

### Working Example 2

Working Example 2 is a modification of Working Example 1. In the dioxaanthanthrene compound of Working Example 2, a substituent A is an alkyl group, substituent R₂ is an alkyl group, and substituents R₁, R₃, and R₄ are hydrogen atoms. Or alternatively, the dioxaanthanthrene compound of Working Example 1 is formed from the 3,9-diphenyl peri-xanthenoxanthene represented by the following structural formula (3), in which, at least an ortho-position and a para-position of two phenyl groups are substituted with a substituent A formed from an alkyl group.

The dioxaanthanthrene compound of Working Example 2 is, specifically, formed from the compound (o-p-iC1Ph-PXX) represented by formula (13). The solubility at 25° C. in 100 g of toluene is as shown in Table 2.

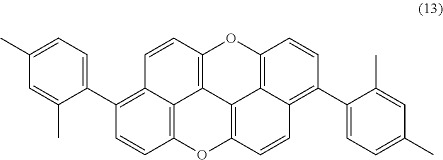

### Working Example 3

Working Example 3 and the below-described Working Examples 4 to 7 relate to the electronic device according to the present disclosure.

The electronic device of Working Example 3, or the electronic device of Working Examples 4 to 7, includes at least

a first electrode,

a second electrode disposed separated from the first electrode, and

an active layer provided from the first electrode to the second electrode,

wherein the active layer is formed from the dioxaanthanthrene compound represented by structural formula (1) or structural formula (3).

Specifically, the electronic device of Working Example 3, or the electronic device of Working Examples 4 to 7, is a three-terminal type electronic device that includes

a first electrode,

a second electrode disposed separated from the first electrode, and

a control electrode; and

an insulating layer,

wherein the control electrode is provided facing a portion of an active layer that is positioned between the first electrode and the second electrode via the insulating layer.

More specifically, the three-terminal type electronic devices of Working Example 3 and the below Working Examples 4 to 6 are field-effect transistors (FETs) in which the current flowing in an active layer from a first electrode to a second electrode is controlled based on the voltage applied to a control electrode, in which the control electrode corresponds to a gate electrode, the first electrode and the second electrode correspond to source/drain electrodes, an insulating layer corresponds to a gate insulating layer film, and the active layer corresponds to a channel formation region.

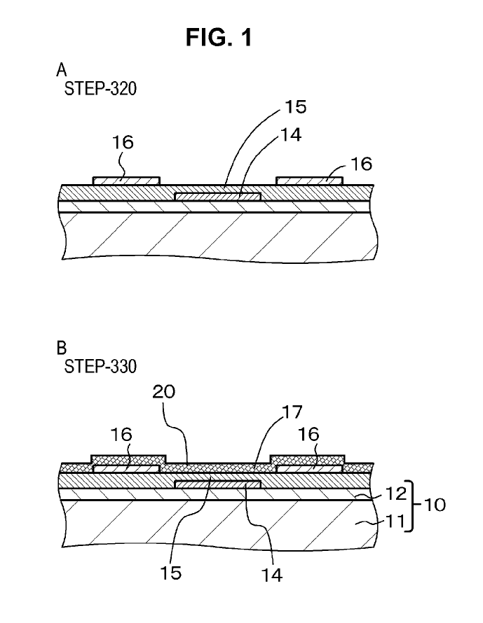

Namely, as illustrated in the schematic partial end view of FIG. 1B, the electronic device of Working Example 3 is a semiconductor device, specifically, a bottom-gate/bottom-contact type field-effect transistor (more specifically, a thin-film transistor (TFT)), which includes

(A) a gate electrode 14 (corresponding to the control electrode) formed on a base 10,

(B) a gate insulating layer 15 (corresponding to an insulating layer) formed on the gate electrode 14 and the base 10,

(C) source/drain electrodes 16 (corresponding to the first electrode and the second electrode) formed on the gate insulating layer 15, and

(D) a channel formation region 17 configured by an active layer 20, which is formed on the gate insulating layer 15 between the source/drain electrodes 16.

An outline of the method for manufacturing the electronic device (field-effect transistor) of Working Example 4 will now be described with reference to FIGS. 1A and 1B, which are schematic partial end views of the base and the like.

### Step-300

First, the gate electrode 14 is formed on the base 10. Specifically, based on a photolithography technique, a resist layer (not illustrated), from which the portion where the gate electrode 14 is to be formed has been removed, is formed on the insulating film 12 including SiO₂ that is formed on the surface of the glass substrate 11. Then, a titanium (Ti) layer (not illustrated) as an adhesion layer and a gold (Au) layer as the gate electrode 14 are successively deposited on the whole face by a vacuum deposition method, after which the resist layer is removed. In this way, based on a so-called lift-off method, the gate electrode 14 can be obtained (refer to FIG. 8A). It is noted that the gate electrode 14 can also be formed on the insulating film 12 including SiO₂ that is formed on the surface of the glass substrate 11 based on a printing method.

### Step-310

Next, the gate insulating layer 15 corresponding to the insulating layer is formed on the base 10 (more specifically, the insulating film 12 formed on the surface of the glass substrate 11) including the gate electrode 14. Specifically, the gate insulating layer 15 that includes SiO₂ is formed on the gate electrode 14 and the insulating film 12 based on a sputtering method. When depositing the gate insulating layer 15, an extraction portion (not illustrated) of the gate electrode 14 can be formed without using a photolithography process by covering a part of the gate electrode 14 with a hard mask.

### Step-320

Then, the source/drain electrodes 16 formed from a 25 nm-thick gold (Au) layer are formed on the gate insulating layer 15 based on a screen printing method (refer to FIG. 1A).

### Step-330

Next, the channel formation region 17 (active layer 20) can be formed on the gate insulating layer 15 and the source/drain electrodes 16 by, specifically, coating and drying a solution obtained by dissolving the dioxaanthanthrene compound represented in structural formula (1) or structural formula (3) in toluene based on a pin coating method (refer to FIG. 1B).

### Step-340

For example, in the manufacture of an image display device, following on from this step, an image display device can be manufactured by forming an image display unit (specifically, an image display unit including an organic electroluminescence element or an electrophoretic display element, a semiconductor light emitting element or the like) based on a known method on or above the thus-obtained TFT, which is an electronic device constituting the control unit (pixel drive circuit) of an image display device. Here, the thus-obtained electronic device constituting the control unit (pixel drive circuit) of an image display device and the electrodes (e.g., pixel electrodes) in the image display unit may be, for example, connected by a connection portion such as a contact hole or a wire. In the below-described Working Example 4 to Working Example 6 as well, an image display device can be obtained by carrying out a similar step after manufacture of the electronic device is completed.

Alternatively, a passivation film (not illustrated) is formed on the whole face. By doing so, a bottom-gate/bottom-contact type semiconductor device (a FET, specifically, a TFT) can be obtained. Alternatively, a passivation film (not illustrated) may be formed on the whole face after patterning the channel formation region 17 and the gate insulating layer 15. This enables the adhesive properties of the active layer 20 and the gate insulating layer 15 to be improved.

### Working Example 4

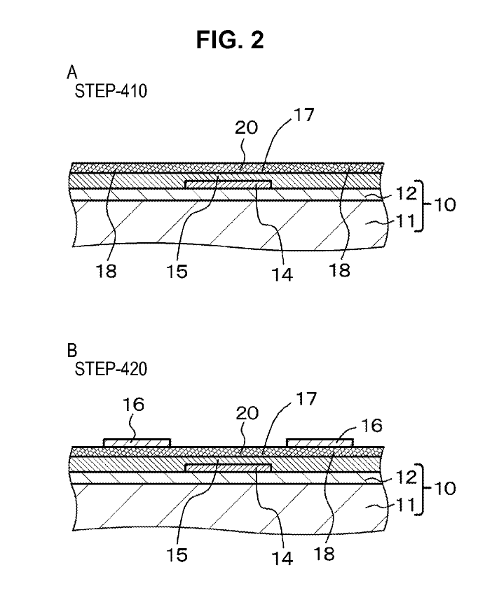

Working Example 4 is a modification of Working Example 3. In Working Example 4, the three-terminal type electronic device is a bottom-gate/top-contact type FET (specifically, a TFT). As illustrated in the schematic partial end view of FIG. 2B, the field-effect transistor of Working Example 4 includes

(A) the gate electrode 14 (corresponding to the control electrode) formed on the base 10,

(B) the gate insulating layer 15 (corresponding to an insulating layer) formed on the gate insulating layer 15 and the base 10,

(C) the channel formation region 17 and the channel formation region extension portion 18 which are formed on the gate insulating layer 15 and are configured by the active layer 20, and

(D) source/drain electrodes 16 (corresponding to the first electrode and the second electrode) formed on the channel formation region extension portion 18.

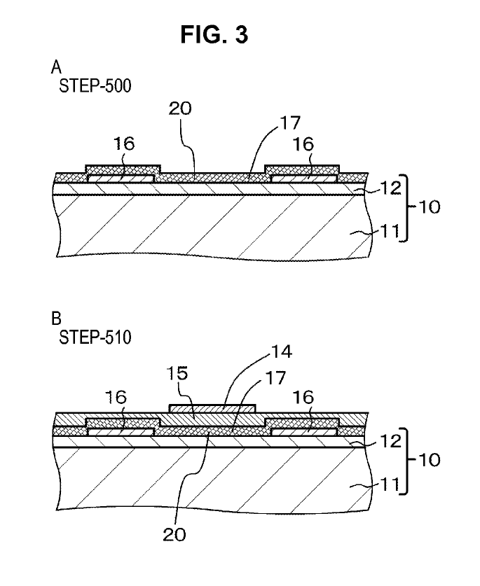

An outline of the method for manufacturing the electronic device (field-effect transistor) of Working Example 5 will now be described with reference to FIGS. 3A and 3B, which are schematic partial end views of the base and the like.

### Step-400

First, the gate electrode 14 is formed on the base 10 in the same manner as in “Step-300” of Working Example 3, and then the gate insulating layer 15 is formed on the base (more specifically, the insulating film 12) including the gate electrode 14 in the same manner as in “Step-310” of Working Example 3.

### Step-410

Next, the active layer 20 is formed on the gate insulating layer 15 in the same manner as in “Step-330” of Working Example 3 (refer to FIG. 2A). In this way, the channel formation region 17 and the channel formation region extension portion 18 can be obtained.

### Step-420

Then, the source/drain electrodes 16 are formed on the channel formation region extension portion 18 so as to sandwich the channel formation region 17 (refer to FIG. 2B). Specifically, a gold (Au) layer is formed as the source/drain electrodes 16 based on a screen printing method in the same manner as “Step-320” of Working Example 3.

### Step-430

Next, the electronic device of Working Example 4 can be completed by carrying out the same step as in “Step-340” of Working Example 3.

### Working Example 5

Working Example 5 is a modification of Working Example 3. In Working Example 5, the three-terminal type electronic device is a top-gate/bottom-contact type FET (specifically, a TFT). As illustrated in the schematic partial end view of FIG. 3B, the field-effect transistor of Working Example 5 includes

(A) source/drain electrodes 16 (corresponding to the first electrode and the second electrode) formed on the base 10,

(B) the channel formation region 17 which is formed on the base 10 between the source/drain electrodes 16 and is configured by the active layer 20,

(C) the gate insulating layer 15 (corresponding to the insulating layer) formed on the source/drain electrodes 16 and the channel formation region 17, and

(D) the gate electrode 14 (corresponding to the control electrode) formed on the gate insulating layer 15.

An outline of the method for manufacturing the electronic device (field-effect transistor) of Working Example 5 will now be described with reference to FIGS. 3A and 3B, which are schematic partial end views of the base and the like.

### Step-500

First, the source/drain electrodes 16 are formed on the insulating film 12 corresponding to the base in the same manner as in “Step-320” of Working Example 3, and then the channel formation region 17 (the active layer 20) is formed on the insulating film 12 including the source/drain electrodes 16 in the same manner as in “Step-330” of Working Example 3 (refer to FIG. 3A).

### Step-510

Next, the gate insulating layer 15 is formed in the same manner as in “Step-310” of Working Example 3. Then, the gate electrode 14 is formed on the portion of the gate insulating layer 15 on the channel formation region 17 in the same manner as in “Step-300” of Working Example 3 (refer to FIG. 3B).

### Step-520

Next, the electronic device of Working Example 5 can be completed by carrying out the same step as in “Step-340” of Working Example 3.

### Working Example 5

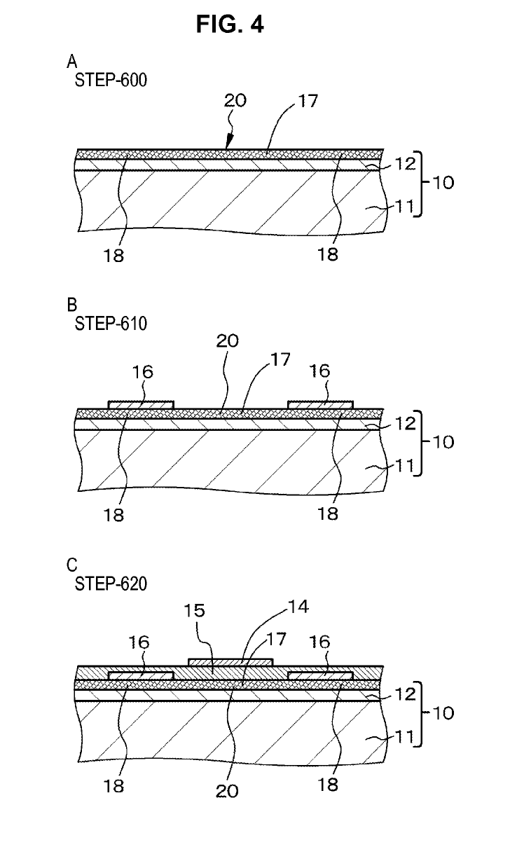

Working Example 6 is a modification of Working Example 3. In Working Example 6, the three-terminal type electronic device is a top-gate/top-contact type FET (specifically, a TFT). As illustrated in the schematic partial end view of FIG. 4C, the field-effect transistor of Working Example 6 includes

(A) the channel formation region 17 and the channel formation region extension portion 18 formed on the base 10 and configured by the active layer 20,

(B) source/drain electrodes 16 (corresponding to the first electrode and the second electrode) formed on the channel formation region extension portion 18,

(C) the gate insulating layer 15 (corresponding to the insulating layer) formed on the source/drain electrodes 16 and the channel formation region 17, and

(D) the gate electrode 14 (corresponding to the control electrode) formed on the gate insulating layer 15.

An outline of the method for manufacturing the electronic device (field-effect transistor) of Working Example 6 will now be described with reference to FIGS. 4A, 4B, and 4C, which are schematic partial end views of the base and the like.

### Step-600

First, the channel formation region 17 and the channel formation region extension portion 18 can be obtained by forming the active layer 20 on the base 10 (more specifically, the insulating film 12) in the same manner as in “Step-330” of Working Example 3 (refer to FIG. 4A).

### Step-610

Next, the source/drain electrodes 16 are formed on the channel formation region extension portion 18 in the same manner as in “Step-320” of Working Example 3 (refer to FIG. 4B).

### Step-620

Then, the gate insulating layer 15 is formed in the same manner as in “Step-310” of Working Example 3. Next, the gate electrode 14 is formed on the portion of the gate insulating layer 15 on the channel formation region 17 in the same manner as in “Step-300” of Working Example 3 (refer to FIG. 4C).

### Step-630

Next, the electronic device of Working Example 6 can be completed by carrying out the same step as in “Step-340” of Working Example 3.

### Working Example 7

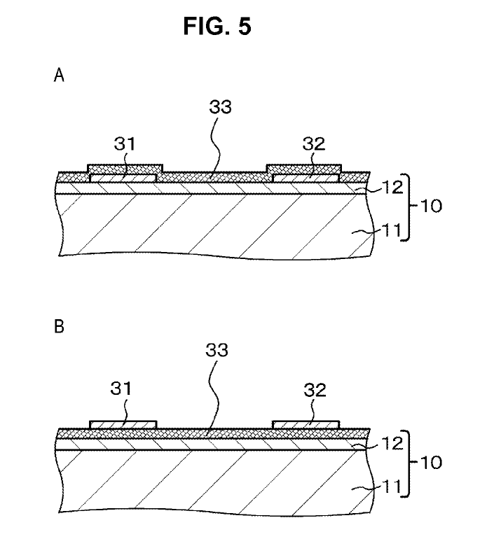

Although Working Example 7 is also a modification of Working Example 3, in Working Example 7 the electronic device is specifically a two-terminal type electronic device, and more specifically, as illustrated in the schematic partial end views of FIG. 5A or 5B, includes

a first electrode 31 and a second electrode 32, and

an active layer 33 formed between the first electrode 31 and the second electrode 32.

It is noted that the active layer 33 includes the dioxaanthanthrene compound described in structural formulas (1) or (3). Further, power is generated by the irradiation of light on the active layer 33. Namely, the electronic device of Working Example 7 functions as a photoelectric conversion element or a solar cell. Alternatively, the electronic device of Working Example 7 functions as a light emitting element in which the active layer 33 emits light due to the application of a voltage to the first electrode 31 and the second electrode 32.

Alternatively, the electronic device of Working Example 7 can also function as a chemical substance sensor including a two-terminal type electronic device. Specifically, when a chemical substance to be detected is adsorbed on the active layer 33, the electric resistance value between the first electrode 31 and the second electrode 32 changes. Therefore, the amount (concentration) of the chemical substance adsorbed on the active layer 33 can be measured by flowing a current between the first electrode 31 and the second electrode 32, or alternatively, applying an appropriate voltage between the first electrode 31 and the second electrode 32, and measuring the electric resistance value of the active layer 33. It is noted that since the chemical substance is in an adsorption equilibrium state at the active layer 33, if the amount (concentration) of the chemical substance in the atmosphere in which the active layer 33 is placed changes, the equilibrium state also changes.

Excluding the above points, basically, the composition and structure of the electronic device of Working Example 7 may be essentially the same as the composition and structure of the electronic device described in Working Example 3 or 4, apart from the point that a control electrode and an insulating layer are not provided. Accordingly, a detailed description thereof will be omitted. The electronic device of Working Example 7 can be obtained by executing the same steps as “Step-320” to “Step-330” of Working Example 3, or alternatively, by executing the same steps as “Step-410” to “Step-420” of Working Example 4.

In the above, although embodiments of the present disclosure were described based on preferred working examples, the present disclosure is not limited to these working examples. The electronic device according to the embodiments of the present disclosure can be, for example, when applied or used in various image display devices or various electronic devices, used as a monolithic integrated circuit in which multiple electronic devices have been integrated on a support member, or each electronic device may be individually separated and used as a discrete component.

For example, in the method for manufacturing the bottom-gate/top-contact type FET (specifically, a TFT) of Working Example 4, the gate insulating layer 15 and the active layer 20 (the channel formation region 17 and the channel formation region extension portion 18) can also be formed based on the below-described phase separation method. Namely, a mixed solution is prepared by uniformly dissolving the dioxaanthanthrene compound represented by structural formula (1) or structural formula (3) and poly(α-methylstyrene), which is an organic insulating material in toluene. Further, a first gate insulating layer that covers the base 10 and the gate electrode 14 is formed in a step similar to “Step-400” of Working Example 4. Specifically, a first gate electrode layer formed from polyvinyl phenol can be obtained by coating a polyvinyl phenol (PVP) solution including an insulating material formed from a photocurable or thermosetting organic material (polymer) and, for example, a cross-linking agent based on a spin coating method on the base 10 and the gate electrode 14, and then heating to 150° C. Next, the above-described mixed solution is coated on the first gate electrode layer based on a spin coating method, and then the obtained coated film is dried in an air atmosphere at 100° C. or more, and preferably 130° C. or more, for 20 to 30 minutes. Consequently, phase separation spontaneously occurs in the coated film, so that a laminated structure of a second gate insulating layer formed from poly(α-methylstyrene) and a dioxaanthanthrene compound layer above that layer is formed. Then, the same steps as in “Step-420” and “Step-430” of Working Example 4 can be carried out. Thus, since there is no contamination of the second gate insulating layer before the dioxaanthanthrene compound layer is formed, the interface between the second gate insulating layer and the dioxaanthanthrene compound layer has a high degree of smoothness, and these layers have a high degree of film thickness precision, an electronic device can be manufactured that has little unevenness in its properties and excellent performance. It is noted that the above-described method for manufacturing an electronic device, or, method for manufacturing an electronic device that is described below, can also be applied to Working Example 1 and Working Examples 5 and 6. Further examples of organic insulating materials other than poly(α-methylstyrene) include a cyclic cycloolefin polymer or a cyclic cycloolefin copolymer. Specific examples of the cyclic cycloolefin polymer or cyclic cycloolefin copolymer include TOPAS (registered trademark, manufactured by Topas Advanced Polymers GmbH), ARTON (registered trademark, manufactured by JSR Corporation), and ZEONOR (registered trademark, manufactured by Zeon Corporation).

Additionally, the present technology may also be configured as below.

### [1] <Dioxaanthanthrene Compound: First Aspect>

A dioxaanthanthrene compound represented by the following structural formula (1),

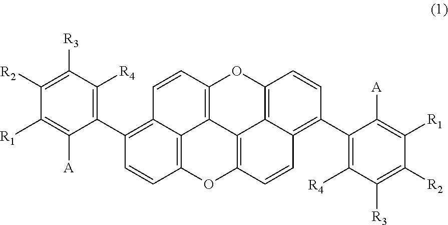

wherein a substituent A is one kind of substituent selected from the group consisting of an alkyl group, a cycloalkyl group, an alkenyl group, an alkynyl group, an aryl group, an arylalkyl group, an aromatic heterocycle, a heterocyclic group, an alkoxy group, a cycloalkoxy group, an aryloxy group, an alkylthio group, a cycloalkylthio group, an arylthio group, an alkoxycarbonyl group, an aryloxycarbonyl group, a sulfamoyl group, an acyl group, an acyloxy group, an amide group, a carbamoyl group, a ureido group, a sulfinyl group, an alkylsulfonyl group, an arylsulfonyl group, an amino group, a halogen atom, a fluorinated hydrocarbon group, a cyano group, a nitro group, a hydroxy group, a mercapto group, and a silyl group including a trimethyl silyl group, and

wherein substituents R₁, R₂, R₃, and R₄ are each independently a hydrogen atom or one kind of substituent selected from the group consisting of an alkyl group, a cycloalkyl group, an alkenyl group, an alkynyl group, an aryl group, an arylalkyl group, an aromatic heterocycle, a heterocyclic group, an alkoxy group, a cycloalkoxy group, an aryloxy group, an alkylthio group, a cycloalkylthio group, an arylthio group, an alkoxycarbonyl group, an aryloxycarbonyl group, a sulfamoyl group, an acyl group, an acyloxy group, an amide group, a carbamoyl group, a ureido group, a sulfinyl group, an alkylsulfonyl group, an arylsulfonyl group, an amino group, a halogen atom, a fluorinated hydrocarbon group, a cyano group, a nitro group, a hydroxy group, a mercapto group, and a silyl group including a trimethyl silyl group.

[2]

The dioxaanthanthrene compound according to [1],

wherein the substituent A is one kind of substituent selected from the group consisting of an alkyl group, an alkenyl group, an aryl group, an arylalkyl group, an aromatic heterocycle, and a halogen atom, and

wherein the substituents R₁, R₂, R₃, and R₄ are each independently a hydrogen atom or one kind of substituent selected from the group consisting of an alkyl group, an alkenyl group, an aryl group, an arylalkyl group, an aromatic heterocycle, and a halogen atom.

[3]

The dioxaanthanthrene compound according to [1],

wherein the substituent A is an alkyl group,

wherein the substituents R₁, R₂, R₃, and R₄ are each independently a hydrogen atom or the same alkyl group as the substituent A, and

wherein a solubility of the dioxaanthanthrene compound represented by structural formula (1) is three times or more with respect to a solubility of a dioxaanthanthrene compound represented by structural formula (2).

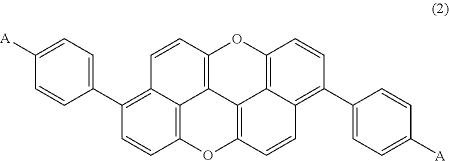

### [4]<Dioxaanthanthrene Compound: Second Aspect>

A dioxaanthanthrene compound formed of 3,9-diphenyl peri-xanthenoxanthene, in which at least an ortho-position of two phenyl groups is substituted with a substituent A,

wherein the substituent A is one kind of substituent selected from the group consisting of an alkyl group, a cycloalkyl group, an alkenyl group, an alkynyl group, an aryl group, an arylalkyl group, an aromatic heterocycle, a heterocyclic group, an alkoxy group, a cycloalkoxy group, an aryloxy group, an alkylthio group, a cycloalkylthio group, an arylthio group, an alkoxycarbonyl group, an aryloxycarbonyl group, a sulfamoyl group, an acyl group, an acyloxy group, an amide group, a carbamoyl group, a ureido group, a sulfinyl group, an alkylsulfonyl group, an arylsulfonyl group, an amino group, a halogen atom, a fluorinated hydrocarbon group, a cyano group, a nitro group, a hydroxy group, a mercapto group, and a silyl group including a trimethyl silyl group.

[5]

The dioxaanthanthrene compound according to [4], wherein an ortho-position of two phenyl groups is substituted with the substituent A.

[6]

The dioxaanthanthrene compound according to [4], wherein an ortho-position and a para-position of two phenyl groups, or an ortho-position and a meta-position of two phenyl groups, are substituted with the substituent A.

### [7]<Electronic Device>

An electronic device including at least:

a first electrode;

a second electrode disposed separated from the first electrode; and

an active layer provided from the first electrode to the second electrode,

wherein the active layer is formed of the dioxaanthanthrene compound according to any one of any one of [1] to [6].

[8]

The electronic device according to [7], including:

the first electrode;

the second electrode disposed separated from the first electrode;

a control electrode; and

an insulating layer,

wherein the control electrode is provided facing a portion of the active layer that is positioned between the first electrode and the second electrode via the insulating layer.

### REFERENCE SIGNS LIST

10 base
11 glass substrate
12 insulating film
14 gate electrode (control electrode)
15 gate insulating layer (insulating layer)
16 source/drain electrodes (first electrode and second electrode)
17 channel formation region
18 channel formation region channel extension portion
20, 33 active layer
31 first electrode
32 second electrode

1. A dioxaanthanthrene compound represented by the following structural formula (1), 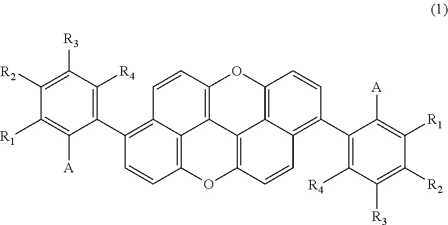
wherein a substituent A is one kind of substituent selected from the group consisting of an alkyl group, a cycloalkyl group, an alkenyl group, an alkynyl group, an aryl group, an arylalkyl group, an aromatic heterocycle, a heterocyclic group, an alkoxy group, a cycloalkoxy group, an aryloxy group, an alkylthio group, a cycloalkylthio group, an arylthio group, an alkoxycarbonyl group, an aryloxycarbonyl group, a sulfamoyl group, an acyl group, an acyloxy group, an amide group, a carbamoyl group, a ureido group, a sulfinyl group, an alkylsulfonyl group, an arylsulfonyl group, an amino group, a halogen atom, a fluorinated hydrocarbon group, a cyano group, a nitro group, a hydroxy group, a mercapto group, and a silyl group including a trimethyl silyl group, and
wherein substituents R₁, R₂, R₃, and R₄ are each independently a hydrogen atom or one kind of substituent selected from the group consisting of an alkyl group, a cycloalkyl group, an alkenyl group, an alkynyl group, an aryl group, an arylalkyl group, an aromatic heterocycle, a heterocyclic group, an alkoxy group, a cycloalkoxy group, an aryloxy group, an alkylthio group, a cycloalkylthio group, an arylthio group, an alkoxycarbonyl group, an aryloxycarbonyl group, a sulfamoyl group, an acyl group, an acyloxy group, an amide group, a carbamoyl group, a ureido group, a sulfinyl group, an alkylsulfonyl group, an arylsulfonyl group, an amino group, a halogen atom, a fluorinated hydrocarbon group, a cyano group, a nitro group, a hydroxy group, a mercapto group, and a silyl group including a trimethyl silyl group.

2. The dioxaanthanthrene compound according to claim 1,
wherein the substituent A is one kind of substituent selected from the group consisting of an alkyl group, an alkenyl group, an aryl group, an arylalkyl group, an aromatic heterocycle, and a halogen atom, and
wherein the substituents R₁, R₂, R₃, and R₄ are each independently a hydrogen atom or one kind of substituent selected from the group consisting of an alkyl group, an alkenyl group, an aryl group, an arylalkyl group, an aromatic heterocycle, and a halogen atom.

3. The dioxaanthanthrene compound according to claim 1,
wherein the substituent A is an alkyl group,
wherein the substituents R₁, R₂, R₃, and R₄ are each independently a hydrogen atom or the same alkyl group as the substituent A, and
wherein a solubility of the dioxaanthanthrene compound represented by structural formula (1) is three times or more with respect to a solubility of a dioxaanthanthrene compound represented by structural formula (2).

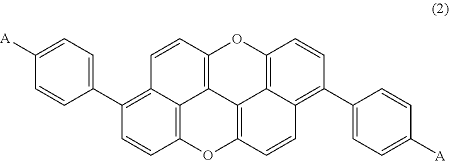

4. A dioxaanthanthrene compound formed of 3,9-diphenyl peri-xanthenoxanthene, in which at least an ortho-position of two phenyl groups is substituted with a substituent A,
wherein the substituent A is one kind of substituent selected from the group consisting of an alkyl group, a cycloalkyl group, an alkenyl group, an alkynyl group, an aryl group, an arylalkyl group, an aromatic heterocycle, a heterocyclic group, an alkoxy group, a cycloalkoxy group, an aryloxy group, an alkylthio group, a cycloalkylthio group, an arylthio group, an alkoxycarbonyl group, an aryloxycarbonyl group, a sulfamoyl group, an acyl group, an acyloxy group, an amide group, a carbamoyl group, a ureido group, a sulfinyl group, an alkylsulfonyl group, an arylsulfonyl group, an amino group, a halogen atom, a fluorinated hydrocarbon group, a cyano group, a nitro group, a hydroxy group, a mercapto group, and a silyl group including a trimethyl silyl group.

5. The dioxaanthanthrene compound according to claim 4, wherein an ortho-position of two phenyl groups is substituted with the substituent A.

6. The dioxaanthanthrene compound according to claim 4, wherein an ortho-position and a para-position of two phenyl groups, or an ortho-position and a meta-position of two phenyl groups, are substituted with the substituent A.

7. An electronic device comprising at least:
a first electrode;
a second electrode disposed separated from the first electrode; and
an active layer provided from the first electrode to the second electrode,
wherein the active layer is formed of the dioxaanthanthrene compound according to any one of claims 1 to 6.

8. The electronic device according to claim 7, comprising:
the first electrode;
the second electrode disposed separated from the first electrode;
a control electrode; and
an insulating layer,
wherein the control electrode is provided facing a portion of the active layer that is positioned between the first electrode and the second electrode via the insulating layer.
